(19) United States
(12) Patent Application Publication
(10) Pub. No.: US 2026/0248406 A1
(43) Pub. Date: Aug. 27, 2026

(54) BIOSIGNAL MEASUREMENT SYSTEM

(71) Applicant: NTT, Inc., Tokyo (JP)

(72) Inventors: Kento Watanabe, Tokyo (JP); Kenichi Matsunaga, Tokyo (JP)

(21) Appl. No.: 19/133,069

(22) PCT Filed: Nov. 29, 2022
(86) PCT No.: PCT/JP2022/043897

§ 371 Date: May 27, 2025

(51) Int. Cl. A61B 5/053; A61B 5/00

(52) U.S. Cl. CPC A61B 5/053; A61B 5/0002; A61B 5/6802; A61B 5/7225; A61B 2562/04

(57) ABSTRACT

A biosignal measurement system includes first and second sensor devices configured to be attachable to right and left side portions of a person. The first sensor device includes a first electrode configured to detect a biopotential, a first amplifier, a first transmitter configured to wirelessly transmit a first modulated signal, a first piezoelectric element configured to receive an elastic wave and configured to convert it into a second modulated signal, a first receiver configured to demodulate the second modulated signal, and a first reference potential generator. The second sensor device includes a second electrode, a second amplifier, a second transmitter, a second piezoelectric element configured to convert a second modulated signal into an elastic wave, a second receiver configured to demodulate the first modulated signal, and a second reference potential generator.

### CROSS-REFERENCE TO RELATED APPLICATIONS

This application is a national phase entry of PCT Application No. PCT/JP2022/043897, filed on Nov. 29, 2022, which application is hereby incorporated herein by reference.

### TECHNICAL FIELD

The present disclosure relates to a biosignal measurement system that measures a biosignal such as an electrocardiogram signal.

### BACKGROUND

In electrocardiogram measurement which is one type of biopotential measurement, a potential difference between electrodes disposed on both left and right sides of the human body is measured. A biosignal measurement system is provided in a compression sportswear, or the like, to be worn by a person. In an example of FIG. 9, a device 301 is mounted on a portion of a compression sportswear 302 corresponding to a torso central portion, and electrodes 304 provided so as to be in contact with left and right waist portions and a device 301 are connected by a wiring 303 laid along the compression sportswear 302 (Non Patent Literature 1).

Attachment of the electrodes to the torso of a person to be measured as in the example of FIG. 9 causes the person to be measured to feel discomfort due to a feeling of pressure and takes a lot of trouble with attachment, which causes the person to be measured to feel a sense of repellency. Thus, as places other than the torso, for example, the four limbs can be considered as places where the electrodes are to be attached. However, in a case where the electrodes are attached to the right hand and the left hand of the person to be measured, or in a case where the electrodes are attached to the right foot and the left foot, a wiring connecting the left and right electrodes is required, which may restrict activity of the person to be measured.

If the wiring can be eliminated, it is possible to reduce discomfort of the person to be measured due to the wiring and eliminate restraint of the body of the person to be measured. In a case where the wiring is eliminated, and the devices are separated, it is important that the left and right devices use a common reference for potential measurement. If the reference potentials do not coincide with each other, measurement accuracy is deteriorated, which may make it difficult to measure a biopotential.

### CITATION LIST

### Non Patent Literature

Non Patent Literature 1: Nahoko Kasai, Takayuki Ogasawara, Hiroshi Nakashima, and Shingo Tsukada, “Development of Functional Textile “hitoe”: Wearable Electrodes for Monitoring Human Vital Signals”, Communication Society Magazine, 2017, Vol. 11, No. 1, pp. 17-23, The Institute of Electronics, Information and Communication Engineers., Online ISSN 2186-0661, <https://doi.org/10.1587/bplus.11.17>

### SUMMARY

### Technical Problem

The present disclosure has been made to solve the above problems, and an object of embodiments of the present disclosure is to provide a biosignal measurement system capable of easily measuring a biopotential in a form in which a wiring is eliminated, and two devices are separated.

### Solution to Problem

A biosignal measurement system according to embodiments of the present disclosure includes: a first sensor device configured to be attached to one of a right side portion and a left side portion of a person to be measured; and a second sensor device configured to be attached to the other of the right side portion and the left side portion, in which the first sensor device includes: a first electrode configured to detect a biopotential of the person to be measured, a first amplification unit configured to amplify the biopotential detected by the first electrode, a first transmission unit configured to modulate a carrier wave according to the biopotential amplified by the first amplification unit and wirelessly transmit a first modulated signal to the second sensor device, a first piezoelectric element configured to receive an elastic wave transmitted from the second sensor device and convert the elastic wave into a second modulated signal, a first reception unit configured to demodulate the second modulated signal to extract information on a biopotential, and a first reference potential generation unit configured to generate a reference potential of the first amplification unit based on the biopotential amplified by the first amplification unit and the biopotential output from the first reception unit, and the second sensor device includes: a second electrode configured to detect a biopotential of the person to be measured, and a second amplification unit configured to amplify the biopotential detected by the second electrode, a second transmission unit configured to modulate a carrier wave according to the biopotential amplified by the second amplification unit, a second piezoelectric element configured to convert a second modulated signal output from the second transmission unit into an elastic wave and transmit the elastic wave to the first sensor device, a second reception unit configured to demodulate the first modulated signal transmitted from the first sensor device to extract information on the biopotential, and a second reference potential generation unit configured to generate a reference potential of the second amplification unit based on the biopotential amplified by the second amplification unit and the biopotential output from the second reception unit.

### Advantageous Effects

According to embodiments of the present disclosure, by connecting the first sensor device and the second sensor device through wireless communication, it is possible to eliminate a wiring connecting the first sensor device and the second sensor device. This can reduce discomfort of the person to be measured due to the wiring and eliminate restraint of the body of the person to be measured. In the embodiments of the present disclosure, by transmitting and receiving the biopotentials detected by the first and second sensor devices to and from each other, the first and second sensor devices can use a common reference potential for potential measurement, so that measurement accuracy of an electrocardiogram signal can be improved. In addition, in the embodiments of the present disclosure, it is possible to reduce a possibility of interference by transmitting information on the biopotential from the first sensor device to the second sensor device by an electric signal and transmitting information on the biopotential from the second sensor device to the first sensor device by an elastic wave.

### BRIEF DESCRIPTION OF THE DRAWINGS

FIG. 1 is a block diagram illustrating a configuration of a biosignal measurement system according to a first embodiment of the present disclosure.

FIG. 2 is a circuit diagram illustrating a configuration of an amplification unit according to the first embodiment of the present disclosure.

FIG. 3 is a block diagram illustrating a configuration of a biosignal measurement system according to a second embodiment of the present disclosure.

FIG. 4 is a block diagram illustrating a configuration of a biosignal measurement system according to a third embodiment of the present disclosure.

FIG. 5 is a block diagram illustrating a configuration of a biosignal measurement system according to a fourth embodiment of the present disclosure.

FIG. 6 is a block diagram illustrating another configuration of the biosignal measurement system according to the fourth embodiment of the present disclosure.

FIG. 7 is a block diagram illustrating a configuration of a biosignal measurement system according to a fifth embodiment of the present disclosure.

FIG. 8 is a block diagram illustrating a configuration example of a computer that implements the biosignal measurement system according to the first to fifth embodiments of the present disclosure.

FIG. 9 is a view illustrating a configuration of a biosignal measurement system in related art.

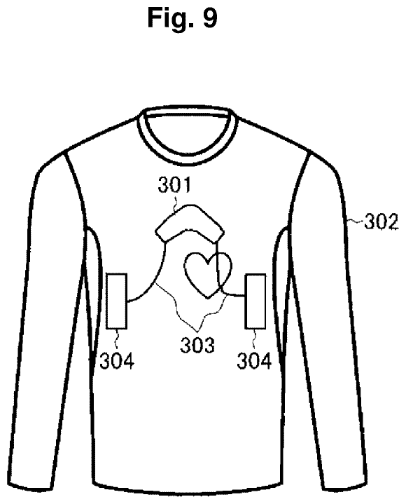

### DETAILED DESCRIPTION OF ILLUSTRATIVE EMBODIMENTS

### First Embodiment

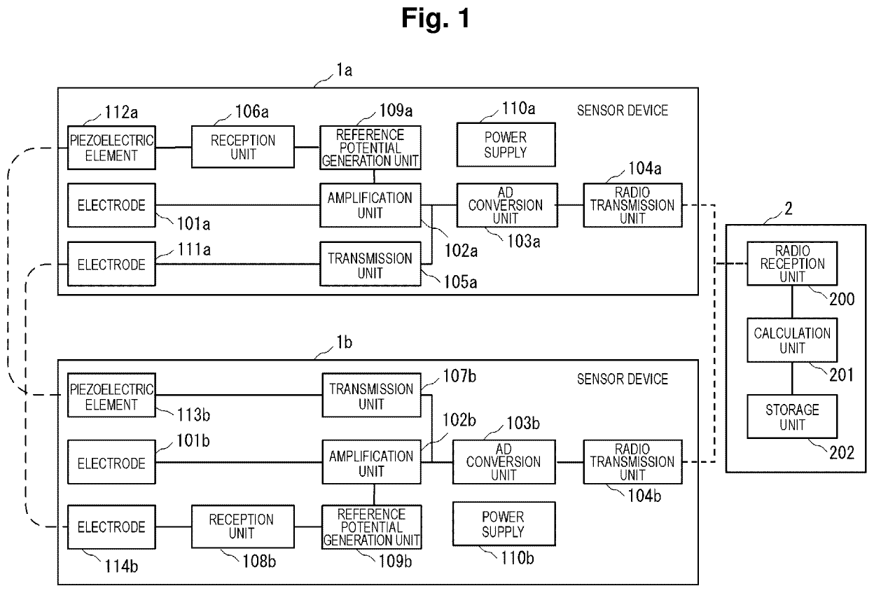

Embodiments of the present disclosure will be described below with reference to the drawings. FIG. 1 is a block diagram illustrating a configuration of a biosignal measurement system according to a first embodiment of the present disclosure. The biosignal measurement system includes a sensor device 1a to be attached to a right side portion of a person to be measured, a sensor device 1b to be attached to a left side portion, and a biosignal generation device 2.

The sensor device 1a includes an electrode 101a in contact with the skin of the right side portion of the person to be measured, an amplification unit 102a that amplifies a biopotential detected by the electrode 101a, an AD conversion unit 103a that converts the amplified biopotential into digital data, a radio transmission unit 104a that wirelessly transmits the digital data output from the AD conversion unit 103a to the biosignal generation device 2, a transmission unit 105a that modulates a carrier wave according to the biopotential amplified by the amplification unit 102a and transmits a modulated signal to the sensor device 1b, a reception unit 106a that demodulates the modulated signal transmitted from the sensor device 1b to extract information on the biopotential, and a reference potential generation unit 109a that generates a reference potential of the amplification unit 102a, a power supply 110a that supplies power to the amplification unit 102a, the AD conversion unit 103a, the radio transmission unit 104a, the transmission unit 105a, the reception unit 106a, and the reference potential generation unit 109a, an electrode 111a in contact with the skin of the right side portion of the person to be measured for transmitting the modulated signal output from the transmission unit 105a to the sensor device 1b via the body of the person to be measured, and a piezoelectric element 112a that receives the elastic wave transmitted from the sensor device 1b and converts the elastic wave into an electric signal.

The sensor device 1b includes an electrode 101b in contact with the skin of the left side portion of the person to be measured, an amplification unit 102b that amplifies the biopotential detected by the electrode 101b, an AD conversion unit 103b that converts the amplified biopotential into digital data, a radio transmission unit 104b that wirelessly transmits the digital data output from the AD conversion unit 103b to the biosignal generation device 2, a transmission unit 107b that modulates a carrier wave according to the biopotential amplified by the amplification unit 102b and transmits a modulated signal to the sensor device 1a, a reception unit 108b that demodulates the modulated signal transmitted from the sensor device 1a to extract information on the biopotential, and a reference potential generation unit 109b that generates a reference potential of the amplification unit 102b, a power supply 110b that supplies power to the amplification unit 102b, the AD conversion unit 103b, the radio transmission unit 104b, the transmission unit 107b, the reception unit 108b, and the reference potential generation unit 109b, a piezoelectric element 113b that converts the modulated signal output from the transmission unit 107b into an elastic wave and transmits the elastic wave to the sensor device 1a via the body of the person to be measured, and an electrode 114b in contact with the skin of the left side portion of the person to be measured for receiving the modulated signal from the sensor device 1a via the body of the person to be measured.

The biosignal generation device 2 includes a radio reception unit 200 that receives the digital data transmitted from the sensor devices 1a and 1b, a calculation unit 201 that calculates an electrocardiogram signal, and a storage unit 202 that stores the electrocardiogram signal calculated by the calculation unit 201.

In a case where the electrocardiogram signal is measured as the biosignal, it is necessary to arrange a plurality of electrodes 101a and 101b at positions sandwiching the heart of the person to be measured. It is conceivable to wear the sensor devices 1a and 1b at at least two positions of the four limbs as measurement sites having a good sense of use for the person to be measured. By adopting such an attachment form of the sensor devices 1a and 1b, it is possible to greatly reduce a feeling of pressure or discomfort due to wearing of a wear, or the like. Note that the biosignal measurement system can be applied not only to the electrocardiogram but also to measurement of myoelectric potential, brain waves, and the like.

In the present embodiment, the sensor devices 1a and 1b have a shape of, for example, a glove, a ring, a sock, a slipper, or a wristband. The person to be measured wears the sensor devices 1a and 1b by putting gloves and rings on the right hand and the left hand, respectively. Alternatively, the person to be measured wears the sensor devices 1a and 1b by wearing socks on the right foot and the left foot, respectively, or by putting on slippers on the right foot and the left foot, respectively. Alternatively, the person to be measured wears the sensor devices 1a and 1b by putting on wristbands on the right hand and the left hand, respectively.

As the electrodes 101a, 101b, 111a, and 114b, electrodes of various materials and configurations can be used. Any electrode such as an Ag/AgCl electrode used in medical applications, a cloth electrode having conductivity, or a metal electrode can be used.

The biopotential detected by the electrodes 101a and 101b is a very weak signal, and thus, it is necessary to amplify the signal by the amplification units 102a and 102b. The amplification units 102a and 102b require high input impedance in order to reduce loss of the biopotential. With an inverting amplifier circuit, a signal-to-noise (SN) ratio of the biopotential is reduced because resistance that determines the input impedance also affects gain setting, and further directly contributes as thermal noise. On the other hand, a non-inverting amplifier circuit has a feature that noise is less likely to increase even in a high input impedance configuration. It is therefore effective to use a non-inverting amplifier circuit as the amplification units 102a and 102b. In addition, low-pass filters may be provided in the amplification units 102a and 102b.

In a case where a non-inverting amplifier circuit is used as the amplification units 102a and 102b, it is important that the two amplification units 102a and 102b use a common reference potential. In the present embodiment, the sensor devices 1a and 1b are not connected by a wiring, and thus, the reference potentials of the amplification units 102a and 102b do not coincide with each other, which may deteriorate measurement accuracy.

Thus, in the present embodiment, in order to improve the measurement accuracy of the electrocardiogram, the amplification units 102a and 102b of the sensor devices 1a and 1b are caused to use a common reference potential Vref by transmitting and receiving information on the biopotential between the sensor devices 1a and 1b.

As will be described later, the biopotential detected by the electrode 101b of the sensor device 1b and amplified by the amplification unit 102b is converted into an elastic wave by the piezoelectric element 113b and transmitted to the sensor device 1a via the body of the person to be measured. The reception unit 106a of the sensor device 1a demodulates the signal transmitted from the sensor device 1b and received by the piezoelectric element 112a to extract information on the biopotential.

The reference potential generation unit 109a of the sensor device 1a generates the reference potential Vref by obtaining an addition average of the biopotential detected by the electrode 101a and amplified by the amplification unit 102a and the biopotential output from the reception unit 106a (the biopotential transmitted from the sensor device 1b).

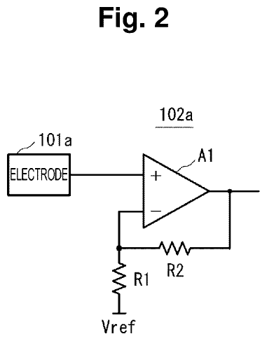

FIG. 2 is a circuit diagram illustrating a configuration example of the amplification unit 102a. The amplification unit 102a includes an operational amplifier A1 and resistors R1 and R2. The reference potential Vref is supplied from the reference potential generation unit 109a to one end of the resistor R1 of the amplification unit 102a.

On the other hand, the biopotential detected by the electrode 101a of the sensor device 1a and amplified by the amplification unit 102a is wirelessly transmitted to the sensor device 1b by the transmission unit 105a and the electrode 111a. The reception unit 108b of the sensor device 1b demodulates the signal transmitted from the sensor device 1a and received by the electrode 114b to extract information on the biopotential.

The reference potential generation unit 109b of the sensor device 1b generates the reference potential Vref by obtaining an addition average of the biopotential detected by the electrode 101b and amplified by the amplification unit 102b and the biopotential output from the reception unit 108b (biopotential transmitted from the sensor device 1a) and supplies the reference potential Vref to the amplification unit 102b. A configuration of the amplification unit 102b is the same as that of the amplification unit 102a. Each of the reference potential generation units 109a and 109b preferably includes, for example, a one-stage operational amplifier.

The AD conversion unit 103a of the sensor device 1a converts the biopotential amplified by the amplification unit 102a into digital data. The radio transmission unit 104a wirelessly transmits the biopotential data output from the AD conversion unit 103a to the biosignal generation device 2.

Similarly, the AD conversion unit 103b of the sensor device 1b converts the biopotential amplified by the amplification unit 102b into digital data. The radio transmission unit 104b wirelessly transmits the biopotential data output from the AD conversion unit 103b to the biosignal generation device 2.

Any wireless communication standards such as carrier communication, Wi-Fi (registered trademark), and Bluetooth (registered trademark) can be applied as the wireless communication standards between the radio transmission units 104a and 104b and the radio reception unit 200 of the biosignal generation device 2. In a case where short-range communication standards such as Bluetooth are adopted, a smartphone, or the like, which is a terminal close to the person to be measured, can be used as the biosignal generation device 2.

Furthermore, in a case where Wi-Fi, or the like, is adopted, a server device, or the like, can be used as the biosignal generation device 2.

The calculation unit 201 of the biosignal generation device 2 calculates a difference between the biopotential transmitted from the sensor device 1a and the biopotential transmitted from the sensor device 1b as an electrocardiogram signal. The electrocardiogram signal is stored in the storage unit 202.

Next, communication between the sensor devices 1a and 1b will be described in more detail. In the present embodiment, human body communication using the body of the person to be measured as a transmission path is used as a method of transmitting and receiving data between the sensor devices 1a and 1b. Power for communication accounts for much of power consumption of the sensor devices 1a and 1b. In spatial propagation using radio waves, signal intensity attenuates in inverse proportion to a square of a propagation distance. On the other hand, in a case of the human body communication, attenuation of a signal is only inversely proportional to the propagation distance. Thus, use of the human body communication enables data transmission with less transmission power. Transmission and reception of data via the human body can contribute to reduction in power consumption. In addition, electric field radiation to an external environment is reduced, so that it is not necessary to adjust a transmission frequency to a specific frequency as in radio wave communication.

The transmission unit 105a of the sensor device 1a modulates a carrier wave according to the biopotential amplified by the amplification unit 102a and transmits the modulated signal from the electrode 111a to the sensor device 1b via the body of the person to be measured. The piezoelectric element 112a of the sensor device 1a receives the elastic wave transmitted from the sensor device 1b via the body of the person to be measured and converts the elastic wave into an electric signal. The reception unit 106a demodulates the modulated signal output from the piezoelectric element 112a to extract information on the biopotential.

The transmission unit 107b of the sensor device 1b modulates a carrier wave according to the biopotential amplified by the amplification unit 102b and outputs a modulated signal to the piezoelectric element 113b. The piezoelectric element 113b converts the modulated signal output from the transmission unit 107b into an elastic wave (ultrasonic wave) and emits the elastic wave to the body of the person to be measured. This elastic wave is transmitted to the sensor device 1a via the body of the person to be measured. The reception unit 108b of the sensor device 1b demodulates the modulated signal transmitted from the electrode 111a of the sensor device 1a and received by the electrode 114b to extract information on the biopotential.

The piezoelectric elements 112a and 113b are elements having a piezoelectric effect, and if a voltage is input, a pressure corresponding to the voltage is generated, and if a pressure is input, a voltage is generated. Examples of a material of the piezoelectric elements 112a and 113b include lead zirconate titanate (PZT) formed of lead titanate and lead zirconate, and an organic piezoelectric thin film.

PZT does not require large power for signal conversion, so that it is possible to reduce power consumption as compared with power consumption in a case where an electric signal is directly transmitted as in communication between the electrodes 111a and 114b. The organic piezoelectric thin film has physical flexibility. Thus, use of the organic piezoelectric thin film as the material of the piezoelectric elements 112a and 113b can enhance adhesion between the sensor devices 1a and 1b and the person to be measured and enables more stable measurement, and thus, is suitable in the present disclosure.

The piezoelectric elements 112a and 113b have a structure in which a piezoelectric body is sandwiched between two electrodes. Normally, the electrodes of the piezoelectric elements 112a and 113b are insulated and protected. In the present embodiment, by not insulating and protecting the electrodes on a side of the person to be measured of the piezoelectric elements 112a and 113b, the electrodes on the side of the person to be measured of the piezoelectric elements 112a and 113b can be utilized as the electrodes 101a and 101b for measuring an electrocardiogram signal. This eliminates the need of separately preparing the electrodes 101a and 101b, so that it can be expected to achieve reduction in size that is important in wearable devices.

A frequency of the biopotential is about 1 kHz. By using a frequency sufficiently higher than the biopotential as a frequency of vibration (elastic wave) generated on the body of the person to be measured by an ultrasonic wave, it is possible to separate signal transmission between the electrodes 111a and 114b and signal transmission between the piezoelectric elements 112a and 113b.

The elastic wave is suitable for human body communication because the elastic wave propagates through tissues such as bone and fresh and travels in the body of the person to be measured. As a frequency is higher, stronger signal attenuation occurs, and thus, in related art, a frequency of 1 MHz to 10 MHz is used as a frequency of the ultrasonic wave, and a frequency of about 1 Hz to 100 kHz is used as a frequency of bone conduction. Thus, also in the present embodiment, it is considered that use within this frequency range is favorable.

The human body communication has an advantage that bidirectional interference with the outside can be reduced because the electric signal and the elastic wave can be confined in the human body. It is therefore possible to prevent information from being wiretapped from the viewpoint of leakage of a biosignal, and thus it is expected to work advantageously.

As described above, in the present embodiment, by connecting the sensor devices 1a and 1b through wireless communication, a wiring connecting the sensor devices 1a and 1b can be eliminated. This can reduce discomfort of the person to be measured due to the wiring and eliminate restraint of the body of the person to be measured. In the present embodiment, by transmitting and receiving the biopotentials detected by the sensor devices 1a and 1b to and from each other, the sensor devices 1a and 1b can use the common reference potential Vref for the potential measurement, so that measurement accuracy of the electrocardiogram signal can be improved.

In the present embodiment, analog wireless communication is performed between the sensor devices 1a and 1b in order to align the reference potential Vref that is no longer settled as a result of the sensor devices being separated. In the analog wireless communication, there is no processing such as digital calculation that causes a delay time. It is therefore possible to implement a circuit configuration that prevents oscillation and unstable operation when the sensor devices 1a and 1b are coupled.

If the frequency of the modulated signal to be transmitted from the sensor device 1a to the sensor device 1b and the frequency of the modulated signal to be transmitted from the sensor device 1b to the sensor device 1a overlap with each other, interference occurs, and the biopotential cannot be demodulated. Thus, in the present embodiment, a possibility of interference can be reduced by using elastic wave communication for one of transmission from the sensor device 1a to the sensor device 1b and transmission from the sensor device 1b to the sensor device 1a.

### Second Embodiment

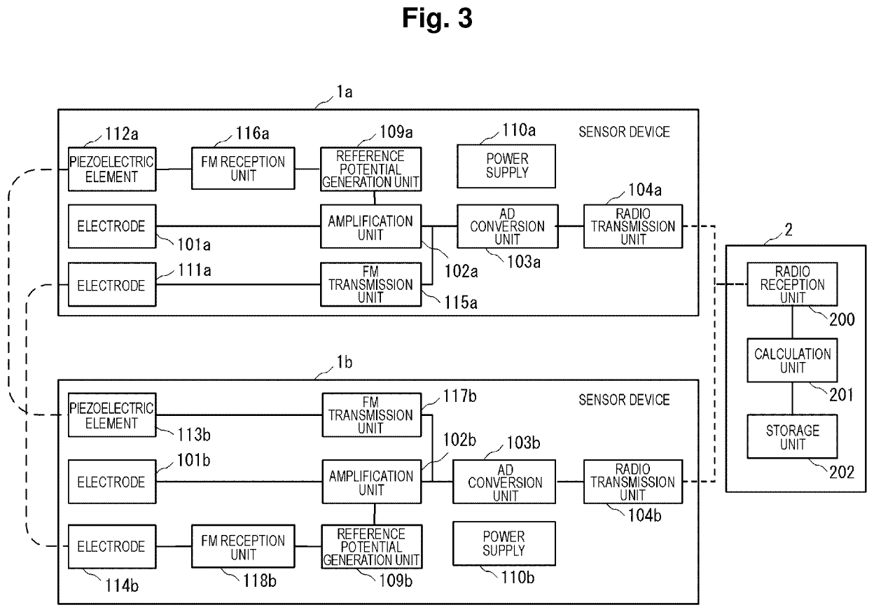

Next, a second embodiment of the present disclosure will be described. FIG. 3 is a block diagram illustrating a configuration of a biosignal measurement system according to a second embodiment of the present disclosure. The present embodiment describes a specific example of the first embodiment.

The sensor device 1a of the present embodiment includes the electrode 101a, the amplification unit 102a, the AD conversion unit 103a, the radio transmission unit 104a, a frequency modulation (FM) transmission unit 115a that frequency-modulates a carrier wave according to the biopotential amplified by the amplification unit 102a and transmits a modulated signal to the sensor device 1b, an FM reception unit 116a that demodulates the modulated signal transmitted from the sensor device 1b to extract information on the biopotential, the reference potential generation unit 109a, the power supply 110a that supplies power to the amplification unit 102a, the AD conversion unit 103a, the radio transmission unit 104a, the FM transmission unit 115a, the FM reception unit 116a, and the reference potential generation unit 109a, the electrode 111a, and the piezoelectric element 112a.

The sensor device 1b of the present embodiment includes the electrode 101b, the amplification unit 102b, the AD conversion unit 103b, the radio transmission unit 104b, an FM transmission unit 117b that frequency-modulates a carrier wave according to the biopotential amplified by the amplification unit 102b and transmits a modulated signal to the sensor device 1a, an FM reception unit 118b that demodulates the modulated signal transmitted from the sensor device 1a to extract information on the biopotential, the reference potential generation unit 109b, the power supply 110b that supplies power to the amplification unit 102b, the AD conversion unit 103b, the radio transmission unit 104b, the FM transmission unit 117b, the FM reception unit 118b, and the reference potential generation unit 109b, the piezoelectric element 113b, and the electrode 114b.

The FM transmission unit 115a of the sensor device 1a frequency-modulates the carrier wave according to the biopotential amplified by the amplification unit 102a and transmits the modulated signal from the electrode 111a to the sensor device 1b via the body of the person to be measured. The FM reception unit 116a demodulates the modulated signal output from the piezoelectric element 112a to extract information on the biopotential.

The FM transmission unit 117b of the sensor device 1b frequency-modulates the carrier wave according to the biopotential amplified by the amplification unit 102b and outputs a modulated signal to the piezoelectric element 113b. The FM reception unit 118b demodulates the modulated signal transmitted from the electrode 111a of the sensor device 1a and received by the electrode 114b to extract information on the biopotential. The other components are the same as those in the first embodiment.

In a case where bidirectional communication between the sensor devices 1a and 1b is performed by an electric signal, it is necessary to change a frequency of a signal transmitted from the sensor device 1a to the sensor device 1b and a frequency of a signal transmitted from the sensor device 1b to the sensor device 1a in order to cause the signals propagate on the same path that is the human body.

The frequency modulation is particularly suitable for a human body, or the like, in which a signal attenuation amount changes depending on a state, because a signal can be transmitted with a maximum amplitude. However, frequencies of carrier waves are changed through frequency modulation, so that it is necessary to prevent the frequencies of the modulated signals from overlapping with each other. In addition, even in a case where the sensor devices 1a and 1b are sufficiently separated so that the carrier frequencies do not overlap with each other, harmonic waves of integral multiples of the carrier frequencies may be superimposed on the modulated signals, and thus, a careful design is required.

In a case where bidirectional communication between the sensor devices 1a and 1b is performed by an electric signal, if the frequencies of modulated signals overlap with each other, there is a possibility that the communication is dominated by a strong wave. In other words, there is a possibility that a received signal is erased by a transmission signal of the own device, and the information transmission fails. For example, in a case where the sensor device 1a oscillates in a 10 MHz band, specifically, oscillates in 9.5 MHz to 10.5 MHz, if the sensor device 1b oscillates such that the frequency of the modulated signal is included between 19 MHz to 21 MHz in addition to the 10 MHz band, information transmission may fail. Harmonics of the third and subsequent orders have the same influence, but high-order harmonics are generally attenuated, and thus, the influence is reduced.

On the other hand, in the present embodiment, a possibility of interference can be reduced by transmitting the information on the biopotential from the sensor device 1a to the sensor device 1b by the electric signal and transmitting the information on the biopotential from the sensor device 1b to the sensor device 1a by the elastic wave. In the present embodiment, even in a case where the carrier frequencies are different from intended design values due to variations in circuit elements or substrate parasitic capacitance, it is possible to transmit a signal without influence.

In addition, in the present embodiment, the carrier frequencies of the sensor devices 1a and 1b forming a pair are not restricted, and thus, a modulation width of frequency modulation can be secured to the maximum, and an SN ratio of the transmission signal can be dramatically improved, so that it is possible to implement biopotential measurement with excellent robustness. Furthermore, in the present embodiment, it is not necessary to set two or more carrier frequencies, and the sensor devices 1a and 1b can perform bidirectional communication using the same carrier frequency, so that it is only necessary to select which of an electrode and a piezoelectric element is used at a contact point between the human body and the device. This eliminates the need for circuit elements for outputting different carrier frequencies, so that it is possible to achieve improvement in mass productivity and cost reduction.

### Third Embodiment

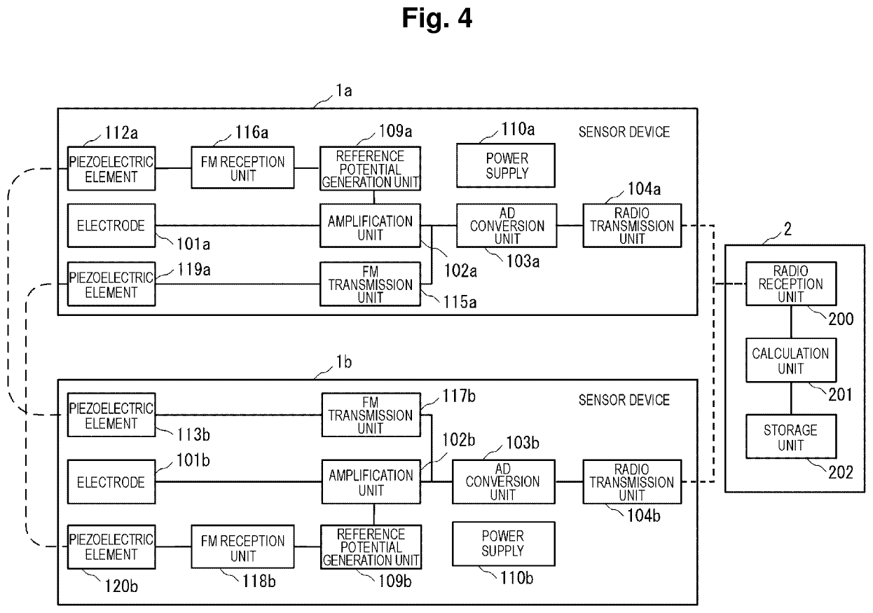

Next, a third embodiment of the present disclosure will be described. FIG. 4 is a block diagram illustrating a configuration of a biosignal measurement system according to the third embodiment of the present disclosure. The sensor device 1a of the present embodiment includes the electrode 101a, the amplification unit 102a, the AD conversion unit 103a, the radio transmission unit 104a, the FM transmission unit 115a, the FM reception unit 116a, the reference potential generation unit 109a, the power supply 110a, and the piezoelectric elements 112a and 119a.

The sensor device 1b of the present embodiment includes the electrode 101b, the amplification unit 102b, the AD conversion unit 103b, the radio transmission unit 104b, the FM transmission unit 117b, the FM reception unit 118b, the reference potential generation unit 109b, the power supply 110b, and the piezoelectric elements 113b and 120b.

The piezoelectric element 119a of the sensor device 1a converts the modulated signal output from the FM transmission unit 115a into an elastic wave (ultrasonic wave) and emits the elastic wave to the body of the person to be measured. This elastic wave is transmitted to the sensor device 1b via the body of the person to be measured.

The piezoelectric element 120b of the sensor device 1b receives the elastic wave transmitted from the sensor device 1a via the body of the person to be measured and converts the elastic wave into an electric signal. The FM reception unit 118b demodulates the modulated signal output from the piezoelectric element 120b to extract information on the biopotential. Other components are similar to those of the second embodiment.

If communication between the sensor devices 1a and 1b is performed by the electric signal, it is expected that the electric signal is superimposed on the biopotential and the SN ratio of the biopotential is deteriorated. In the frequency modulation, a signal is basically transmitted with a maximum amplitude. Thus, the modulated signal that becomes noise becomes close to, for example, 3V with respect to the biopotential of about 1 mV, which causes a potential difference of 3000 times with respect to the biopotential.

The biopotential is a signal having a frequency of about 1 kHz, and thus, if the modulated signal is set to a sufficiently high frequency, it is possible to appropriately separate the biopotential and the modulated signal by filtering. However, the modulated signal has stronger attenuation as the frequency is higher, and it is required to lower a transmission output by setting the frequency to a lower frequency in order to lower the power. Thus, a trade-off occurs between signal quality of the biopotential and the frequency of the modulated signal.

On the other hand, in the present embodiment, the modulated signal is not superimposed on the biopotential by using the elastic wave for both transmission from the sensor device 1a to the sensor device 1b and transmission from the sensor device 1b to the sensor device 1a, and thus, the SN ratio of the biopotential is not deteriorated, and the biosignal measurement can be performed with low power. However, in the present embodiment, the advantage of using the same carrier frequency described in the second embodiment is lost, and it is necessary to use different carrier frequencies in the sensor devices 1a and 1b. In addition, it is necessary to make the frequency of the modulated signal transmitted from the sensor device 1a to the sensor device 1b different from the frequency of the modulated signal transmitted from the sensor device 1b to the sensor device 1a.

Similarly to the first and second embodiments, by not insulating and protecting the electrode on the side of the person to be measured of the piezoelectric element 112a or 119a in the sensor device 1a, the electrode on the side of the person to be measured of the piezoelectric element 112a or 119a can be utilized as the electrode 101a for measuring an electrocardiogram signal. In addition, by not insulating and protecting the electrode on the side of the person to be measured of the piezoelectric element 113b or 120b in the sensor device 1b, the electrode on the side of the person to be measured of the piezoelectric element 113b or 120b can be utilized as the electrode 101b for measuring an electrocardiogram signal.

### Fourth Embodiment

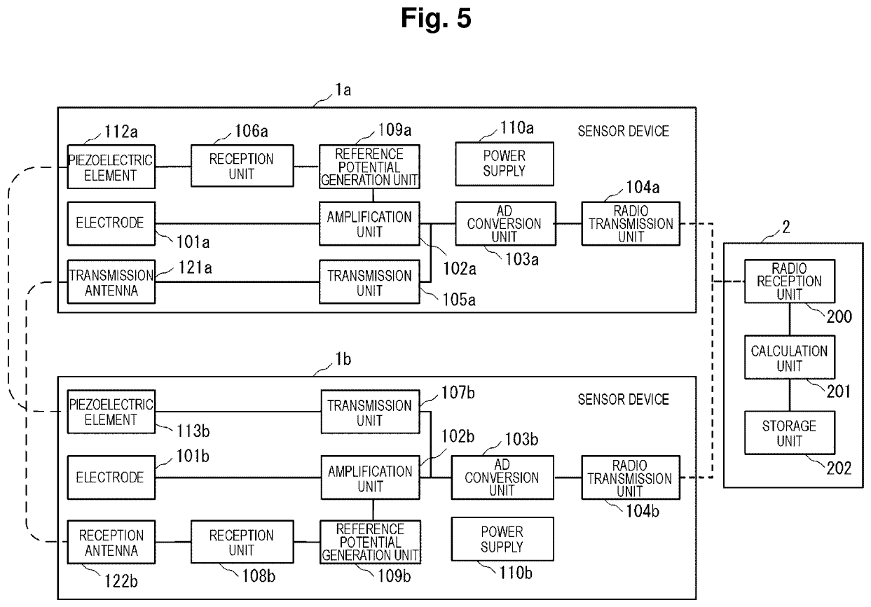

In the first to third embodiments, bidirectional communication between the sensor devices 1a and 1b is performed by human body communication, but wireless communication by radio waves and elastic waves may be performed. FIG. 5 is a block diagram illustrating a configuration of a biosignal measurement system according to a fourth embodiment of the present disclosure.

In the configuration of FIG. 5, a transmission antenna 121a is provided instead of the electrode 111a of FIG. 1, and a reception antenna 122b is provided instead of the electrode 114b. The transmission unit 105a of the sensor device 1a may modulate the carrier wave according to the biopotential amplified by the amplification unit 102a and transmit the modulated signal from the transmission antenna 121a to the sensor device 1b. The reception unit 108b of the sensor device 1b demodulates the modulated signal transmitted from the sensor device 1a and received by the reception antenna 122b to extract information on the biopotential.

In the first embodiment, the piezoelectric element 113b of the sensor device 1b emits an elastic wave to the body of the person to be measured. On the other hand, in the present embodiment, the piezoelectric element 113b only requires to emit an elastic wave (ultrasonic wave) to a space toward the sensor device 1a. The piezoelectric element 112a of the sensor device 1a receives the elastic wave transmitted from the sensor device 1b via the space and converts the elastic wave into an electric signal.

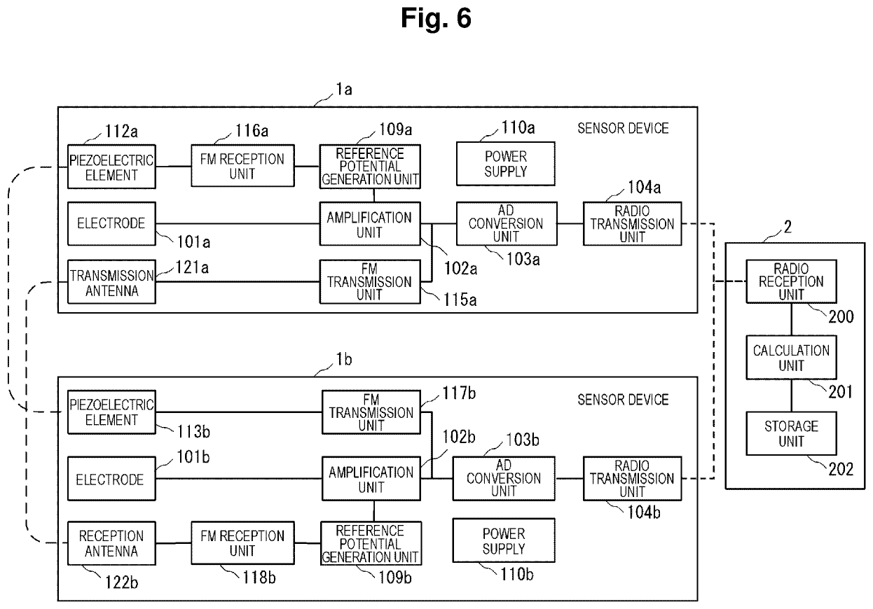

The present embodiment may be applied to the second embodiment. A configuration in this case is illustrated in FIG. 6. In the configuration of FIG. 6, the FM transmission unit 115a of the sensor device 1a only requires to frequency-modulate the carrier wave according to the biopotential amplified by the amplification unit 102a and transmit the modulated signal from the transmission antenna 121a to the sensor device 1b. The FM reception unit 118b of the sensor device 1b demodulates the modulated signal transmitted from the sensor device 1a and received by the reception antenna 122b to extract information on the biopotential. The operation of the piezoelectric elements 112a and 113b is the same as the operation in the configuration of FIG. 5.

The present embodiment may be applied to the third embodiment. In this case, the piezoelectric element 119a of the sensor device 1a only requires to emit an elastic wave (ultrasonic wave) to a space toward the sensor device 1b. The piezoelectric element 120b of the sensor device 1b receives the elastic wave transmitted from the sensor device 1a via the space and converts the elastic wave into an electric signal. The operation of the piezoelectric elements 112a and 113b is the same as the operation in the configuration of FIG. 5.

### Fifth Embodiment

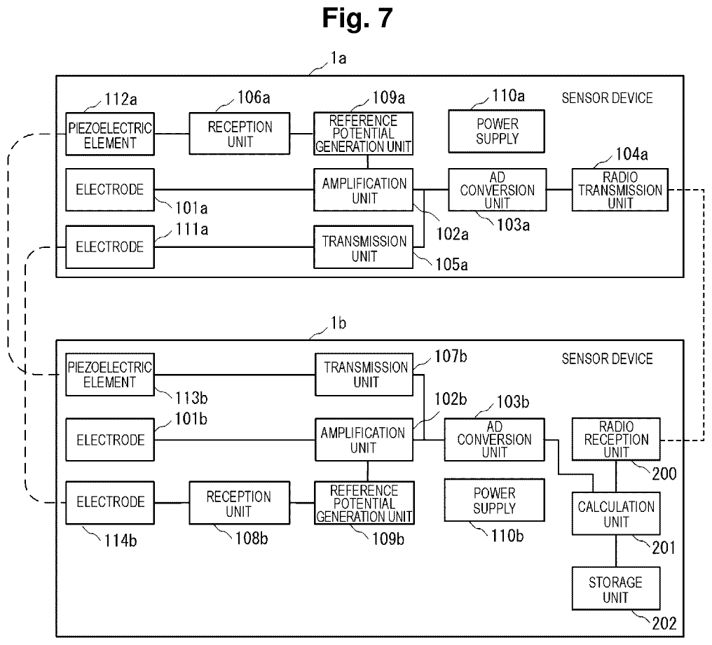

In the first to fourth embodiments, the biosignal generation device 2 is provided separately from the sensor devices 1a and 1b, but the configuration of the biosignal generation device 2 may be mounted on either one of the sensor devices 1a and 1b. FIG. 7 is a block diagram illustrating a configuration of a biosignal measurement system according to a fifth embodiment of the present disclosure.

In the configuration of FIG. 7, the radio transmission unit 104b of the sensor device 1b is unnecessary. The radio reception unit 200 provided in the sensor device 1b receives the biopotential data transmitted from the sensor device 1a. The calculation unit 201 calculates a difference between the biopotential transmitted from the sensor device 1a and the biopotential output from the AD conversion unit 103b as an electrocardiogram signal. The electrocardiogram signal is stored in the storage unit 202.

In the present embodiment, it is not necessary to provide the biosignal generation device 2 separately from the sensor devices 1a and 1b, and thus, the person to be measured does not need to carry the biosignal generation device 2, so that it is possible to improve user-friendliness of the person to be measured.

In the example of FIG. 7, the configuration of the biosignal generation device 2 is provided in the sensor device 1b, but it goes without saying that the configuration of the biosignal generation device 2 may be provided in the sensor device 1a. The configuration of FIG. 7 illustrates an example in which the present embodiment is applied to the configuration of FIG. 1, but the present embodiment may be applied to the configurations of FIGS. 3 to 6.

In the first to fifth embodiments, the sensor device 1a may be attached to the left side portion of the person to be measured, and the sensor device 1b may be attached to the right side portion.

Further, while in the first to fifth embodiments, the electrodes 101a, 101b, 111a, and 114b and the piezoelectric elements 112a, 113b, 119a, and 120b are in contact with the skin of the person to be measured, the electrodes 101a, 101b, 111a, and 114b and the piezoelectric elements 112a, 113b, 119a, and 120b may have a non-contact configuration in which they are not in contact with the skin. With the non-contact configuration, the sensor devices 1a and 1b can be worn from above the clothing of the person to be measured, so that it is possible to further reduce burden on the person to be measured and implement biosignal measurement that does not interfere with daily activities.

In the non-contact configuration, capacitive coupling is formed between the electrodes 101a, 101b, 111a, and 114b and the skin, so that it is possible to transmit and receive a biosignal and an electric signal even in a case where there is clothing between the electrodes 101a, 101b, 111a, and 114b and the skin. In addition, the elastic wave transmits vibration. Thus, even in a case where there is clothing between the piezoelectric elements 112a, 113b, 119a, and 120b and the skin, vibration is transmitted to the skin, so that the elastic wave can be transmitted and received. By appropriately adopting the non-contact configuration, even in a case where different attachment portions are required for each need of the person to be measured, it is possible to flexibly cope with the requirement, and it is possible to expand availability of the biosignal measurement system.

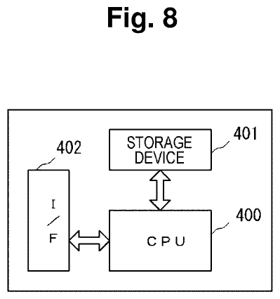

The calculation unit 201 and the storage unit 202 described in the first to fifth embodiments can be implemented by a computer including a central processing unit (CPU), a storage device, and an interface, and a program for controlling these hardware resources. A configuration example of this computer is illustrated in FIG. 8.

The computer includes a CPU 400, a storage device 401, and an interface device (I/F) 402. Hardware, or the like, of the radio reception unit 200 is connected to the I/F 402. A program for implementing the method of the present disclosure is stored in the storage device 401. The CPU 400 executes the processing described in the first to fifth embodiments according to a program stored in the storage device 401. In addition, at least part of the calculation unit 201 may be configured by hardware logic such as a field-programmable gate array (FPGA).

Some or all of the above-described embodiments may be described as the following supplementary notes, but are not limited to the following.

(Supplementary note 1) A biosignal measurement system according to the present disclosure includes: a first sensor device configured to be attached to one of a right side portion and a left side portion of a person to be measured, and a second sensor device configured to be attached to the other of the right side portion and the left side portion, in which the first sensor device includes: a first electrode configured to detect a biopotential of the person to be measured; a first amplification unit configured to amplify the biopotential detected by the first electrode, a first transmission unit configured to modulate a carrier wave according to the biopotential amplified by the first amplification unit and wirelessly transmit a first modulated signal to the second sensor device, a first piezoelectric element configured to receive an elastic wave transmitted from the second sensor device and convert the elastic wave into a second modulated signal, a first reception unit configured to demodulate the second modulated signal to extract information on a biopotential, and a first reference potential generation unit configured to generate a reference potential of the first amplification unit based on the biopotential amplified by the first amplification unit and the biopotential output from the first reception unit, and the second sensor device includes: a second electrode configured to detect a biopotential of the person to be measured, a second amplification unit configured to amplify the biopotential detected by the second electrode, a second transmission unit configured to modulate a carrier wave according to the biopotential amplified by the second amplification unit, a second piezoelectric element configured to convert a second modulated signal output from the second transmission unit into an elastic wave and transmit the elastic wave to the first sensor device, a second reception unit configured to demodulate the first modulated signal transmitted from the first sensor device to extract information on the biopotential, and a second reference potential generation unit configured to generate a reference potential of the second amplification unit based on the biopotential amplified by the second amplification unit and the biopotential output from the second reception unit.

(Supplementary note 2) A biosignal measurement system according to the present disclosure includes: a first sensor device configured to be attached to one of a right side portion and a left side portion of a person to be measured, and a second sensor device configured to be attached to the other of the right side portion and the left side portion, in which the first sensor device includes: a first electrode configured to detect a biopotential of the person to be measured, a first amplification unit configured to amplify the biopotential detected by the first electrode, and a first transmission unit configured to modulate a carrier wave according to the biopotential amplified by the first amplification unit, a first piezoelectric element configured to convert a first modulated signal output from the first transmission unit into an elastic wave and transmit the elastic wave to the second sensor device, a second piezoelectric element configured to receive the elastic wave transmitted from the second sensor device and convert the elastic wave into a second modulated signal, a first reception unit configured to demodulate the second modulated signal to extract information on a biopotential, and a first reference potential generation unit configured to generate a reference potential of the first amplification unit based on the biopotential amplified by the first amplification unit and the biopotential output from the first reception unit, the second sensor device includes: a second electrode configured to detect a biopotential of the person to be measured, a second amplification unit configured to amplify the biopotential detected by the second electrode, a second transmission unit configured to modulate a carrier wave according to the biopotential amplified by the second amplification unit, a third piezoelectric element configured to convert a second modulated signal output from the second transmission unit into an elastic wave and transmit the elastic wave to the first sensor device, a fourth piezoelectric element configured to receive the elastic wave transmitted from the first sensor device and convert the elastic wave into a first modulated signal, a second reception unit configured to demodulate the first modulated signal output from the fourth piezoelectric element to extract information on a biopotential, and a second reference potential generation unit configured to generate a reference potential of the second amplification unit based on the biopotential amplified by the second amplification unit and the biopotential output from the second reception unit, and a frequency of the first modulated signal is different from a frequency of the second modulated signal.

(Supplementary note 3) In the biosignal measurement system according to Supplementary note 1, the first sensor device further includes a third electrode that transmits the first modulated signal output from the first transmission unit to the second sensor device via the body of the person to be measured, the second sensor device further includes a fourth electrode that receives the first modulated signal from the first sensor device via the body of the person to be measured, the second piezoelectric element radiates the elastic wave converted from the second modulated signal output from the second transmission unit to the body of the person to be measured, and the first piezoelectric element receives the elastic wave from the second sensor device via the body of the person to be measured and converts the elastic wave into the second modulated signal.

(Supplementary note 4) The biosignal measurement system according to Supplementary note 1, in which the first sensor device further includes a transmission antenna that wirelessly transmits the first modulated signal output from the first transmission unit to the second sensor device, the second sensor device further includes a reception antenna that receives the first modulated signal transmitted from the first sensor device, the second piezoelectric element radiates the elastic wave converted from the second modulated signal output from the second transmission unit to a space toward the first sensor device, and the first piezoelectric element receives the elastic wave from the second sensor device via the space and converts the elastic wave into the second modulated signal.

(Supplementary note 5) In the biosignal measurement system according to Supplementary note 2, the first piezoelectric element radiates the elastic wave converted from the first modulated signal output from the first transmission unit to the body of the person to be measured, the fourth piezoelectric element receives the elastic wave from the first sensor device via the body of the person to be measured and converts the elastic wave into the first modulated signal, the third piezoelectric element radiates the elastic wave converted from the second modulated signal output from the second transmission unit to the body of the person to be measured, and the second piezoelectric element receives the elastic wave from the second sensor device via the body of the person to be measured and converts the elastic wave into the second modulated signal.

(Supplementary note 6) In the biosignal measurement system according to Supplementary note 2, the first piezoelectric element radiates the elastic wave converted from the first modulated signal output from the first transmission unit to a space toward the second sensor device, the fourth piezoelectric element receives the elastic wave from the first sensor device via the space and converts the elastic wave into the first modulated signal, the third piezoelectric element radiates the elastic wave converted from the second modulated signal output from the second transmission unit to the space toward the first sensor device, and the second piezoelectric element receives the elastic wave from the second sensor device via the space and converts the elastic wave into the second modulated signal.

(Supplementary note 7) The biosignal measurement system according to Supplementary note 1 or 2, further including a biosignal generation device, in which the first sensor device further includes a third transmission unit configured to wirelessly transmit data of the biopotential amplified by the first amplification unit to the biosignal generation device, the second sensor device further includes a fourth transmission unit configured to wirelessly transmit data of the biopotential amplified by the second amplification unit to the biosignal generation device, and the biosignal generation device further includes a third reception unit configured to receive the data of the biopotential transmitted from the first sensor device and the data of the biopotential transmitted from the second sensor device, and a calculation unit configured to calculate an electrocardiogram signal of the person to be measured based on the biopotential transmitted from the first sensor device and the biopotential transmitted from the second sensor device.

(Supplementary note 8) In the biosignal measurement system according to Supplementary note 1 or 2, the first sensor device further includes a third transmission unit configured to wirelessly transmit data of the biopotential amplified by the first amplification unit to the second sensor device, and the second sensor device further includes a third reception unit configured to receive the data of the biopotential transmitted from the third transmission unit, and a calculation unit configured to calculate an electrocardiogram signal of the person to be measured based on the biopotential transmitted from the first sensor device and the biopotential amplified by the second amplification unit.

### Industrial Applicability

The present disclosure can be applied to a technique for measuring a biosignal.

### REFERENCE SIGNS LIST

1a, 1b Sensor device
2 Biosignal generation device
101a, 101b, 111a, 114b Electrode
102a, 102b Amplification unit
103a, 103b AD conversion unit
104a, 104b Radio transmission unit
105a, 107b Transmission unit
106a, 108b Reception unit
109a, 109b Reference potential generation unit
110a, 110b Power supply
112a, 113b, 119a, 120b Piezoelectric element
115a, 117b FM transmission unit
116a, 118b FM reception unit
121a Transmission antenna
122b Reception antenna

1-8. (canceled)

9. A biosignal measurement system comprising:
a first sensor device configured to be attached to one of a right side portion and a left side portion of a person to be measured; and
a second sensor device configured to be attached to the other of the right side portion and the left side portion,
wherein the first sensor device includes:
a first electrode configured to detect a biopotential of the person to be measured;
a first amplifier configured to amplify the biopotential detected by the first electrode;
a first transmitter configured to modulate a carrier wave according to the biopotential amplified by the first amplifier and wirelessly transmit a first modulated signal to the second sensor device;
a first piezoelectric element configured to receive an elastic wave transmitted from the second sensor device and convert the elastic wave into a second modulated signal;
a first receiver configured to demodulate the second modulated signal to extract information on a biopotential; and
a first reference potential generator configured to generate a reference potential of the first amplifier based on the biopotential amplified by the first amplifier and the biopotential extracted by the first receiver, and
the second sensor device includes:
a second electrode configured to detect a biopotential of the person to be measured;
a second amplifier configured to amplify the biopotential detected by the second electrode;
a second transmitter configured to modulate a carrier wave according to the biopotential amplified by the second amplifier;
a second piezoelectric element configured to convert a second modulated signal output from the second transmitter into an elastic wave and transmit the elastic wave to the first sensor device;
a second receiver configured to demodulate the first modulated signal transmitted from the first sensor device to extract information on the biopotential; and
a second reference potential generator configured to generate a reference potential of the second amplifier based on the biopotential amplified by the second amplifier and the biopotential extracted by the second receiver.

10. A biosignal measurement system comprising:
a first sensor device configured to be attached to one of a right side portion and a left side portion of a person to be measured;
a second sensor device configured to be attached to the other of the right side portion and the left side portion,
wherein the first sensor device includes:
a first electrode configured to detect a biopotential of the person to be measured;
a first amplifier configured to amplify the biopotential detected by the first electrode;
a first transmitter configured to modulate a carrier wave according to the biopotential amplified by the first amplifier;
a first piezoelectric element configured to convert a first modulated signal output from the first transmitter into an elastic wave and transmit the elastic wave to the second sensor device;
a second piezoelectric element configured to receive the elastic wave transmitted from the second sensor device and convert the elastic wave into a second modulated signal;
a first receiver configured to demodulate the second modulated signal to extract information on a biopotential; and
a first reference potential generator configured to generate a reference potential of the first amplifier based on the biopotential amplified by the first amplifier and the biopotential extracted by the first receiver,
the second sensor device includes:
a second electrode configured to detect a biopotential of the person to be measured;
a second amplifier configured to amplify the biopotential detected by the second electrode;
a second transmitter configured to modulate a carrier wave according to the biopotential amplified by the second amplifier;
a third piezoelectric element configured to convert a second modulated signal output from the second transmitter into an elastic wave and transmit the elastic wave to the first sensor device;
a fourth piezoelectric element configured to receive the elastic wave transmitted from the first sensor device and convert the elastic wave into a first modulated signal;
a second receiver configured to demodulate the first modulated signal output from the fourth piezoelectric element to extract information on a biopotential; and
a second reference potential generator configured to generate a reference potential of the second amplifier based on the biopotential amplified by the second amplifier and the biopotential extracted by the second receiver, and
a frequency of the first modulated signal is different from a frequency of the second modulated signal.

11. The biosignal measurement system according to claim 9, wherein
the first sensor device further includes a third electrode that transmits the first modulated signal output from the first transmitter to the second sensor device via a body of the person to be measured,
the second sensor device further includes a fourth electrode that receives the first modulated signal from the first sensor device via the body of the person to be measured,
the second piezoelectric element radiates the elastic wave converted from the second modulated signal output from the second transmitter to the body of the person to be measured, and
the first piezoelectric element receives the elastic wave from the second sensor device via the body of the person to be measured and converts the elastic wave into the second modulated signal.

12. The biosignal measurement system according to claim 9, wherein
the first sensor device further includes a transmission antenna that wirelessly transmits the first modulated signal output from the first transmitter to the second sensor device,
the second sensor device further includes a reception antenna that receives the first modulated signal transmitted from the first sensor device,
the second piezoelectric element radiates the elastic wave converted from the second modulated signal output from the second transmitter to a space toward the first sensor device, and
the first piezoelectric element receives the elastic wave from the second sensor device via the space and converts the elastic wave into the second modulated signal.

13. The biosignal measurement system according to claim 10, wherein
the first piezoelectric element radiates the elastic wave converted from the first modulated signal output from the first transmitter to the body of the person to be measured,
the fourth piezoelectric element receives the elastic wave from the first sensor device via the body of the person to be measured and converts the elastic wave into the first modulated signal,
the third piezoelectric element radiates the elastic wave converted from the second modulated signal output from the second transmitter to the body of the person to be measured, and
the second piezoelectric element receives the elastic wave from the second sensor device via the body of the person to be measured and converts the elastic wave into the second modulated signal.

14. The biosignal measurement system according to claim 10, wherein
the first piezoelectric element radiates the elastic wave converted from the first modulated signal output from the first transmitter to a space toward the second sensor device,
the fourth piezoelectric element receives the elastic wave from the first sensor device via the space and converts the elastic wave into the first modulated signal,
the third piezoelectric element radiates the elastic wave converted from the second modulated signal output from the second transmitter to a space toward the first sensor device, and
the second piezoelectric element receives the elastic wave from the second sensor device via the space and converts the elastic wave into the second modulated signal.

15. The biosignal measurement system according to claim 9, further comprising:
a biosignal generation device, wherein
the first sensor device further includes a third transmitter configured to wirelessly transmit data of the biopotential amplified by the first amplifier to the biosignal generation device,
the second sensor device further includes a fourth transmitter configured to wirelessly transmit data of the biopotential amplified by the second amplifier to the biosignal generation device, and
the biosignal generation device includes:
a third receiver configured to receive the data of the biopotential transmitted from the first sensor device and the data of the biopotential transmitted from the second sensor device; and
a calculator configured to calculate an electrocardiogram signal of the person to be measured based on the biopotential data transmitted from the first sensor device and the biopotential data transmitted from the second sensor device.

16. The biosignal measurement system according to claim 9, wherein
the first sensor device further includes:
a third transmitter configured to wirelessly transmit data of the biopotential amplified by the first amplifier to the second sensor device, and
the second sensor device further includes:
a third receiver configured to receive the data of the biopotential transmitted from the third transmitter; and
a calculator configured to calculate an electrocardiogram signal of the person to be measured based on the biopotential data transmitted from the first sensor device and the biopotential amplified by the second amplifier.

17. The biosignal measurement system according to claim 10, further comprising:
a biosignal generation device, wherein
the first sensor device further includes a third transmitter configured to wirelessly transmit data of the biopotential amplified by the first amplifier to the biosignal generation device,
the second sensor device further includes a fourth transmitter configured to wirelessly transmit data of the biopotential amplified by the second amplifier to the biosignal generation device, and
the biosignal generation device includes:
a third receiver configured to receive the data of the biopotential transmitted from the first sensor device and the data of the biopotential transmitted from the second sensor device; and
a calculator configured to calculate an electrocardiogram signal of the person to be measured based on the biopotential data transmitted from the first sensor device and the biopotential data transmitted from the second sensor device.

18. The biosignal measurement system according to claim 10, wherein
the first sensor device further includes:
a third transmitter configured to wirelessly transmit data of the biopotential amplified by the first amplifier to the second sensor device, and
the second sensor device further includes:
a third receiver configured to receive the data of the biopotential transmitted from the third transmitter; and
a calculator configured to calculate an electrocardiogram signal of the person to be measured based on the biopotential data transmitted from the first sensor device and the biopotential amplified by the second amplifier.

19. A biosignal measurement system comprising:
a first wearable device configured to be attached to a first limb of a person;
a second wearable device configured to be attached to a second limb of the person;
wherein the first wearable device comprises:
a first electrode configured to detect a first biopotential;
a first amplifier configured to amplify the first biopotential;
a first transmitter configured to wirelessly transmit first biopotential data; and
a first elastic wave receiver configured to receive an elastic wave from the second wearable device;
wherein the second wearable device comprises:
a second electrode configured to detect a second biopotential;
a second amplifier configured to amplify the second biopotential;
a second transmitter configured to wirelessly transmit second biopotential data; and
an elastic wave generator configured to transmit an elastic wave to the first wearable device;
wherein the first and second wearable devices are configured to use a common reference potential for biopotential measurement based on exchanging biopotential information.

20. The biosignal measurement system of claim 19, wherein the first wearable device is configured to be attached to a right hand or a right foot of the person, and the second wearable device is configured to be attached to a left hand or a left foot of the person.

21. The biosignal measurement system of claim 19, wherein the first wearable device and the second wearable device each have a shape selected from the group consisting of a glove, a ring, a sock, a slipper, and a wristband.

22. The biosignal measurement system of claim 19, wherein the first transmitter is configured to transmit the first biopotential data using human body communication via the person's body.

23. The biosignal measurement system of claim 19, wherein the elastic wave generator is configured to transmit the elastic wave as an ultrasonic wave.

24. The biosignal measurement system of claim 19, further comprising a biosignal generation device configured to:
receive the first biopotential data from the first wearable device;
receive the second biopotential data from the second wearable device; and
calculate an electrocardiogram signal based on a difference between the first biopotential data and the second biopotential data.

25. The biosignal measurement system of claim 19, wherein the first wearable device further comprises a first reference potential generator configured to generate the common reference potential based on the first biopotential and information extracted from the elastic wave received from the second wearable device.

26. The biosignal measurement system of claim 19, wherein the first elastic wave receiver and the elastic wave generator each comprise a piezoelectric element.

27. The biosignal measurement system of claim 26, wherein the piezoelectric element of at least one of the first elastic wave receiver or the elastic wave generator is configured to also function as the first electrode or the second electrode, respectively.

28. The biosignal measurement system of claim 19, wherein the first and second wearable devices are configured to operate without a wired connection between them.
